(19) United States
(12) Patent Application Publication
(10) Pub. No.: US 2024/0374620 A1
(43) Pub. Date: Nov. 14, 2024

(54) INTRANASAL COMPOSITION AND METHOD

(71) Applicant: SinuCare Natural Health Care Corporation, Huntington Beach, CA (US)

(72) Inventors: Kathy Yu-Syken, Huntington Beach, CA (US); Jeffrey Charles Raber, Rancho Palos Verdes, CA (US); Sytze Warren Elzinga, Lakewood, CO (US)

(21) Appl. No.: 18/661,370

(22) Filed: May 10, 2024

Related U.S. Application Data
(60) Provisional application No. 63/466,194, filed on May 12, 2023.

(51) Int. Cl. A61K 31/00; A61K 9/00; A61K 31/047; A61K 36/53; A61K 36/80; A61K 47/26

(52) U.S. Cl. CPC A61K 31/658; A61K 9/0043; A61K 31/047; A61K 36/53; A61K 36/80; A61K 47/26

(57) ABSTRACT

A composition comprising cannabigerol, xylitol, and at least one of adaptogens, such as mullein leaf and/or holy basil is disclosed. The composition may be formulated as a sinus/nasal rinse or a nasal spray, and can be used to alleviate, relieve and/or treat the symptoms relating to sinus or nasal mucosal inflammation.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The disclosed invention is in the field of composition for alleviating or treatment of mucosal inflammation.

### Description of the Related Art

Sinus and nasal mucosal inflammation may be caused by food or environmental allergies, infections (viral, bacterial, fungal), laryngeal pharyngeal acid and/or bile reflux, chemical irritation (including side effects of nasally delivered medication), and climate change. The current treatment options for mucosal inflammation include saline sinus rinse and medicated nasal sprays. Plain saline sinus rinse is known to mechanically rinse away allergens and particulate matter that may irritate the sensitive nasal mucous membranes, however, does not provide anti-inflammatory or anti-microbial benefits. Medicated nasal sprays may dry out the mucous membranes of the nose, which can cause unpleasant side effects for patients and other users of the rinse. Therefore, there is a need for a composition for relieving or treating sinus and nasal mucosal inflammation without the unpleasant side effects.

### SUMMARY

Presently disclosed is a composition comprising cannabigerol, xylitol, and at least one of mullein leaf and holy basil, wherein the composition is for intranasal administration.

In some embodiments, the cannabigerol is in an amount of about 1 mg to about 20 mg. In some embodiments, the composition further comprises a carrier. In some embodiments, the composition further comprises polysorbate 80. In some embodiments, the composition further comprises sodium chloride and/or sodium bicarbonate, and an emulsifier. In some embodiments, the composition further comprises an additional cannabinoid. In some embodiments, the composition further comprises cannabidiol. In some embodiments, the composition further comprises tetrahydrocannabinol. In some embodiments, the composition further comprises cannabichromene. In some embodiments, the composition is in a powder form. In some embodiments, the composition is configured to form a nasal/sinus rinse. In some embodiments, the composition is a nasal spray.

Disclosed is a method of alleviating symptoms of chronic rhinosinusitis comprising administering the composition of any one of claims 1 to 12 to a subject experiencing chronic rhinosinusitis symptoms.

### DETAILED DESCRIPTION OF EMBODIMENTS

Provided herein is a composition for intranasal delivery of cannabinoids. In some embodiments, the cannabinoid may be cannabigerol (CBG). The composition may be formulated as a nasal/sinus rinse or a nasal spray. In some embodiments, the formulation comprises a solid mixture, for example, a powder mixture. The solid mixture can be added to water or saline solution to form a nasal/sinus rinse or a nasal spray. The composition may be used to ameliorate, alleviate, or treat the symptoms of chronic rhinosinusitis, such as sinus and nasal mucosal inflammation, and to provide anti-microbial protection of sensitive nasal mucous membranes.

### Composition

Cannabigerol (CBG) is one of many cannabinoid compounds found in the plant genus Cannabis. CBG is a decarboxylated form of cannabigerolic acid (CBGA), which is the precursor of all cannabinoid compounds. Since most of the plant's produced CBGA is converted into other cannabinoid acids, such as tetrahydrocannabinol acid (THCA) and cannabidiolic acid (CBDA), the amount of CBGA in a cannabis plant is normally only about 1% with trace amounts of CBG being present due to natural decarboxylation. A larger amount of CBGA can be derived from hemp plants, especially in cultivars that have been bred to produce more CBGA/CBG. As the cannabis plant only produces cannabinoid acids they provide a good source of raw material that can be further processed, via decarboxylation, into neutral cannabinoids. Alternative processing methods, such as fermentation and other cell culture-based techniques, are also good sources of cannabinoid acids as raw materials for decarboxylation.

CBG, like CBD, is non-psychotropic and non-intoxicating. The IUPAC name of CBG is 2-[(2E)-3,7-Dimethylocta-2,6-dienyl]-5-pentyl-benzene-1,3-diol, which has the following structure:

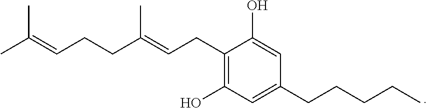 [SMILES: CCCCCc1cc(O)c(C/C=C(\C)CCC=C(C)C)c(O)c1]

Prior studies have shown that CBG may be useful for reducing inflammation associated with inflammatory bowel disease, reducing intraocular pressure (treating glaucoma), reducing the growth of cancer cells and tumors, treating bladder dysfunctions, treating neurodegenerative conditions (such as Huntington's disease), treating conditions attributing to neuro-inflammation, addressing mood imbalance such as depression or anxiety, alleviating pain from musculoskeletal origin, treating bacterial infections, and stimulating appetite. In addition to being an anti-inflammatory and anti-microbial agent, CBG may be useful in reducing and controlling viral, bacterial, and fungal infections.

In some embodiments, the composition comprises CBG, xylitol, and sodium chloride. The combination of CBG and xylitol in saline may reduce transmission of virus or other pathologic microorganisms by decreasing viral load in the nasal cavity and nasopharynx in addition to decreasing viral adhesion to mucous membranes. This would then make mucous membranes in nasal and sinus cavities less susceptible to viral and other microorganism penetration when moisturized with xylitol and when inflammation is decreased from CBG.

In some embodiments, the composition comprises about 1 mg to about 25 mg of CBG. In some embodiments, the composition contains about 1 mg to about 20 mg, about 1 mg to about 15 mg, about 5 mg to about 25 mg, about 5 mg to about 20 mg, about 5 mg to about 15 mg, about 5 mg to about 10 mg, or about 10 mg to about 20 mg of CBG, or any value in between the ranges listed above, including end points. In some embodiments, the composition may contain about 1 mg CBG, about 5 mg CBG, about 10 mg CBG, about 15 mg CBG, or about 20 mg CBG. In some embodiments, CBG may be present in the composition in an amount of about 1 mg, about 2 mg, about 4 mg, about 6 mg, about 8 mg, about 10 mg, about 12 mg, about 14 mg, about 16 mg, about 18 mg, about 20 mg.

In some embodiments, xylitol may be present in the composition in an amount of about 1000 mg to about 4000 mg, about 1000 mg to about 3500 mg, about 1000 mg to about 3000 mg, about 1000 mg to about 2500 mg, about 1000 mg to about 2000 mg of xylitol, about 1000 mg to about 1500 mg, about 1500 mg to about 4000 mg, about 1500 mg to about 3500 mg, about 1500 mg to about 3000 mg, about 1500 mg to about 2500 mg, about 1500 mg to about 2000 mg, about 2000 mg to about 4000 mg, about 2000 mg to about 3500 mg, about 2500 mg to about 4000 mg and any value in between the ranges listed, including end points. In some embodiments xylitol may be present in the composition in an amount of about 1000 mg, about 1500 mg, about 2000 mg, about 2500 mg, about 3000 mg, about 3500 mg, or about 4000 mg.

In some embodiments, the composition may contain sodium chloride in the amount of about 950 mg to about 1900 mg, about 1,000 mg to about 1,800 mg, about 900 mg to about 1900 mg, about 850 to about 1,950 mg, about 800 to about 2,000 mg, and any value in between the ranges listed, including endpoints. In some embodiments, sodium chloride may be present in the composition in an amount of about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, or about 1900 mg.

In some embodiments, the composition may further comprise an emulsifier, such as polysorbate 80, Quillaja extract, propylene glycol, randomly methylated cyclodextrin (dissolved state), and randomly methylated cyclodextrin (solid state). In some embodiments. the composition may further comprise polysorbate 80. Emulsifiers may aid the formulation by improving interactions with the mucosal lining of interest, and further may also improve bioavailability. The emulsifier may be present in the composition in the amount of about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 80 mg, about 30 mg to about 70 mg, about 30 mg to about 60 mg, about 30 mg to about 50 mg, about 30 mg to about 40 mg, about 40 mg to about 80 mg, about 40 mg to about 70 mg, about 40 mg to about 60 mg, about 40 mg to about 50 mg, about 50 mg to about 80 mg, about 50 mg to about 70 mg, about 50 mg to about 60 mg, about 60 mg to about 80 mg, about 60 mg to about 70 mg, about 70 mg to about 80 mg, and any value in between the ranges listed, including endpoints. In some embodiments, the emulsifier may be present in the composition in an amount of about 20 mg, about 30 mg, about 40 mg, about 50 mg, about 60 mg, about 70 mg, about 80 mg.

In some embodiments, the composition may also include sodium bicarbonate. Sodium bicarbonate can serve as a buffering agent to make the pH of a formulation suitable for nasal mucous membranes such that it does not cause a burning sensation present in traditional saline sinus rinses. In some embodiments, the buffering agent provides a pH range of between about 5 and about 7, between about 5 and about 6, between about 6 and about 7, between about 4.9 and about 6.1, between about 4.8 and about 6.2, between about 4.7 and about 6.3, between about 4.6 and about-6.4, between about 4.5 and about 6.5, and any value in between the ranges listed, including end points. In some embodiments, sodium bicarbonate may be present in the composition in an amount of between about 490 and about 510 mg, between about 480 and about 520 mg, between about 470 and about 530 mg, between about 460 and about 540 mg, between about 450 and about 550 mg, between about 440 and about 560 mg, between about 430 and about 570 mg, between about 420 and about 580 mg, between about 410 and about 590 mg, between about 400 and about 600 mg, and any value in between the ranges listed, including endpoints. In some embodiments, the amount of sodium bicarbonate may be about 400 mg, about 450 mg, about 500 mb, about 550 mg, or about 600 mg.

In some embodiments, the composition further comprises one or more adaptogens. Adding one or more adaptogens to CBG can further provide synergistic effects in relieving the symptoms associated with chronic rhinosinusitis and reducing mucosal inflammations. Non-limiting examples of adaptogens that can be used includes mullein leaf (Verbascum) and holy basil, a.k.a. Tulsi (Ocimum tenuflorium or Ocimum sanctum). Therefore, in some embodiments, the composition further comprises mullein leaf (Verbascum). Mullein leaf may improve mucociliary clearance so that contaminants can continue to be expelled from the nasal cavity in an efficient manner. In some embodiments, the composition further comprises holy basil, a.k.a. Tulsi (Ocimum tenuflorium or Ocimum sanctum).

Holy Basil, a.k.a. Tulsi may synergistically offer additional anti-bacterial and anti-microbial protection. Our testing also suggests that the composition results in complementary reduction of stress and anxiety, increased focus, and improved memory and cognition. The addition of adaptogens may further increase bioavailability and efficacy of CBG via transnasal absorption.

In some embodiments, mullein leaf verbascum may be present in the composition in the amount of about 1 mg to about 200 mg, about 1 mg to about 100 mg, about 1 mg to about 50 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 50 mg to about 200 mg, about 50 mg to about 150 mg, about 50 mg to about 100 mg, about 100 mg to about 200 mg, about 100 mg to about 150 mg, and any value in between the ranges listed, including endpoints. In some embodiments, mullein leaf verbascum may be present in the composition in an amount of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg.

In some embodiments, the amount of holy basil in the composition can range from about 1 mg to about 300 mg, about 1 mg to about 200 mg, about 1 mg to about 100 mg, about 1 mg to about 50 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, and any value in between the ranges listed, including endpoints. In some embodiments, the holy Basi ocimum may be present in the composition in an amount of about 1 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 200 mg, about 210 mg, about 220 mg, about 230 mg, about 240 mg, about 250 mg, about 260 mg, about 270 mg, about 280 mg, about 290 mg, about 300 mg. In some embodiments, the composition may further comprise one or more other cannabinoids, such as CBD, THC, or CBC. In some embodiments, the composition is formulated with about 1 mg to about 25 mg CBG and about 1 mg to about 25 mg CBD. For example, the amount of CBG in each dose may be about 1 mg, about 5 mg, about 10 mg, or about 20 mg, and the amount of CBD in each dose may be about 1 mg, about 5 mg, about 10 mg, or about 20 mg.

In some embodiments, the composition is formulated with about 1 mg to about 25 mg CBG and about 1 mg to about 25 mg THC. For example, the amount of CBG in each dose may be about 1 mg, about 5 mg, about 10 mg, or about 20 mg, and the amount of THC in each dose may be about 1 mg, about 5 mg, about 10 mg, or about 20 mg.

In some embodiments, the composition is formulated with about 1 mg to about 25 mg CBG and about 1 mg to about 25 mg CBC. For example, the amount of CBG in each dose may be about 1 mg, about 5 mg, about 10 mg, or about 20 mg, and the amount of CBC in each dose may be about 1 mg, about 5 mg, about 10 mg, or about 20 mg.

In some embodiments, the composition may further comprise eucalyptol, camphor, menthol, or a combination thereof to enhance the smell of the formulation.

In some examples, the composition comprises a formulation that includes at least a plurality of xylitol, sodium chloride, sodium bicarbonate, mullein leaf verbascum (mullein leaf powder), holy basil ocimum, CBG, and a carrier (e.g., an emulsion such as polysorbate 80). In some embodiments, the formulation comprises CBG, polysorbate 80, xylitol, sodium chloride, sodium bicarbonate, and mullein leaf verbascum. In some embodiments, the formulation comprises CBG, polysorbate 80, xylitol, sodium chloride, sodium bicarbonate, and holy basil ocimum.

The composition may be a solid mixture, such as a powder mixture or a tablet/pellet. The solid mixture may be configured to provide one dose of the composition. In some examples, the CBG can make up between about 0.05 and about 0.5 wt %, between about 0.1 and about 0.3 wt %, between about 0.1 and about 0.25 wt %, between about 0.1 and about 0.2 wt %, between about 0.15 and about 0.25 wt % of the composition as a whole, and any value in between the ranges listed, including endpoints.

In some examples, polysorbate 80 can make up between about 0.6 and about 0.9 wt %, between 0.65 and about 0.85 wt %, between about 0.7 and about 0.85 wt %, between about 0.75 and about 0.85 wt % of the composition as a whole, and any value in between the ranges listed, including endpoints.

In some embodiments, xylitol may make up between about 50 and about 75 wt %, between about 55 and about 70 wt %, between about 55 and about 65 wt % of the composition as a whole, and any value in between the ranges listed including endpoints.

In some embodiments, the sodium chloride may make up between about 20 and about 40 wt %, between about 25 and about 35 wt %, between about 28 and about 32 wt %, between about 32 and about 38 wt % of the composition as a whole, and any value in between the ranges listed including endpoints.

In some embodiments, the sodium bicarbonate may make up between about 5 and about 20 wt %, between about 8 and about 15 wt % o, between about 8 and about 12 wt %, between about 9 and about 15 wt % of the composition as a whole, and any value in between the ranges listed including endpoints.

In some embodiments, the mullein leaf verbascum may make up between about 0.05-0.9 wt %, between about 0.1 and about 0.9 wt %, between about 0.1 and about 0.8 wt %, between about 0.1 and about 0.7 wt %, between about 0.15 and about 0.5 wt %, between about 0.15 and about 0.3 wt % of the composition as a whole, and any value in between the ranges listed, including endpoints.

In some embodiments, the holy basil ocimum may make up between about 0.05 and about 1 wt %, between about 0.1 and about 0.9 wt %, between about 0.1 and about 0.8 wt %, between about 0.1 and about 0.7 wt %, between about 0.15 and about 0.5 wt %, between about 0.15 and about 0.3 wt % of the composition as a whole, and any value in between the ranges listed including endpoints.

### Method of Forming the Composition

As CBG is a crystalline solid that may not readily dissolve in water, a pre-formulation including CBG and a carrier could be made. The carrier may be an emulsifier, such as polysorbate 80, which allows CBG to be dissolved in polysorbate 80 (using heat as needed). Polysorbate 80 is a non-ionic surfactant and emulsifier that is available as pharmaceutical grade and is a thick water-soluble yellow liquid. In some embodiments, polysorbate 80 can be used as a carrier to enhance the formulation and assist in dissolution of the lipophilic CBG into water. The pre-formulation should be homogeneous after mixing.

In some embodiments, the CBG and the carrier (e.g., emulsifier) may form a pre-mix. The ratio of polysorbate 80 to CBG may be from 1:1 to 100:1, such as 1:1, 2:1, 4:1, 6:1, 8:1, 10:1, 20:1, 30:1, 40:1, 50:1, 60:1, 70:1, 80:1, 90:1, or 100:1. In some embodiments, the ratio of polysorbate 80 to CBG is 1:4.

Xylitol, sodium chloride, and sodium carbonate were mixed together before adding the pre-formulation of CBG and the carrier to the mixture. Appropriate amount of pre-formulation was used to mix with Xylitol, sodium chloride and sodium carbonate to form powders that can later be dissolved in appropriate amount of water to form a nasal/sinus rinse or nasal spray.

### Formulations

In some embodiments, the composition disclosed herein may be formulated as shelf-stable powders or tablet/pellet, which can be mixed in distilled or bottled drinking water to form a nasal/sinus rinse. The nasal/sinus rinse may be formulated for use once per day or twice per day. In some embodiments, the composition may be formulated as a nasal spray, which could be used more frequently such as 3-5 times per day. In some embodiments, the composition described above may be mixed with about 7 oz to about 9 oz, or about 7 oz to about 8 oz, or about 8 oz to about 9 oz of water to form an aqueous formulation, for example, a nasal/sinus rinse formulation. In other embodiments, the composition described above may be mixed with about 1.5 oz to about 2 oz, about 1.5 oz to about 3.0 oz, about 1.5 oz to about 4 oz, about 1.5 oz to about 5 oz, about 2 oz to about 3 oz, about 2 oz to about 4 oz, about 2 oz to about 5 oz, about 3 oz to about 5 oz, about 3 oz to about 4 oz, or about 4 oz to about 5 oz of water to form an aqueous formulation, for example, a nasal spray formulation.

For example, an aqueous solution formulation may be formed by dissolving the composition in 8 oz (approximately 29.57 mL) of liquid (e.g., water). The concentration of CBG in the 8 oz aqueous formulation may be between about 0.03 and about 0.8 mg/mL of aqueous solution, between about 0.05 and about 0.7 mg/mL, between about 0.1 and about 0.7 mg/mL of aqueous solution, between about 0.2 and about 0.6 mg/mL, between about 0.25 and about 0.5 mg/mL of aqueous solution, and any value in between the ranges listed, including endpoints.

The concentration of xylitol in the 8 oz aqueous formulation may be between about 30 and about 140 mg/mL of aqueous solution, between about 50 and about 135 mg/mL of aqueous solution, between about 60 and about 120 mg/mL of aqueous solution, between about 70 and about 130 mg/mL of aqueous solution, and any value in between the ranges listed, including endpoints.

The concentration of sodium chloride in the 8 oz aqueous formulation may be between about 15 and about 80 mg/mL of aqueous solution, between about 30 and about 65 mg/mL of aqueous solution, between about 20 and about and about 60 mg/mL of aqueous solution, between about 25 and about 70 mg/mL of aqueous solution, between about 40 and about 70 mg/mL of aqueous solution, and any value in between the ranges listed, including endpoints.

The concentration of emulsifier in the 8 oz aqueous formulation may be between about 0.2 and about 1 mg/mL of aqueous solution, between about 0.2 and about 0.7 mg/mL of aqueous solution, between about 0.25 and about 0.6 mg/mL of aqueous solution, between about 0.25 and about 0.5 mg/mL of aqueous solution, between about 0.3 and about 0.6 mg/mL of aqueous solution, between about 0.3 and about 0.5 mg/mL of aqueous solution, and any value in between the ranges listed, including endpoints.

The concentration of sodium bicarbonate in the 8 oz aqueous formulation may be between about 15 and about 20 mg/mL of aqueous solution, between about 15 and about 18 mg/mL of aqueous solution, between about 16 and about 20 mg/mL of aqueous solution, between about 16 and about 18 mg/mL of aqueous solution, and any value in between the ranges listed, including endpoints.

The concentration of mullein leaf verbascum in the 8 oz aqueous formulation may be between about 0.1 and about 10 mg/mL of aqueous solution, between about 0.1 and about 7 mg/mL of aqueous solution, between about 0.2 and about 5 mg/ml of aqueous solution, between about 0.2 and about 1 mg/mL of aqueous solution, between about 0.2 and about 0.8 mg/mL of aqueous solution, and any value in between the ranges listed, including endpoints.

The concentration of holy basil ocimum in the 8 oz aqueous formulation may be between about 0.1 and about 15 mg/mL of aqueous solution, between about 0.1 and about 10 mg/mL of aqueous solution, between about 0.2 and about 6 mg/ml of aqueous solution, between about 0.2 and about 1 mg/mL of aqueous solution, between about 0.2 and about 0.8 mg/mL of aqueous solution, and any value in between the ranges listed, including endpoints.

### Method of Treatment

Some embodiments provide a method for alleviating, relieving, or treating symptoms of chronic rhinosinusitis by administering the composition disclosed herein to a subject experiencing chronic rhinosinusitis symptoms. In some embodiments, the composition includes CBG, xylitol, and sodium chloride. In some embodiments, the composition includes CBG, xylitol, sodium chloride, and sodium bicarbonate. In some embodiments, the composition includes CBG, polysorbate 80, xylitol, sodium chloride, and sodium bicarbonate. In some embodiments, any of the composition above may further include holy basil ocimum, mullein leaf verbascum, or both holy basil ocimum and mullein leaf Verbascum. In some embodiments, the composition may consist essentially of CBG, polysorbate 80, xylitol, sodium chloride, and sodium bicarbonate, and one or more adaptogen. In some embodiments, the adaptogen may include holy basil ocimum and/or mullein leaf verbascum.

In some embodiments, Chronic rhinosinusitis symptoms may include sinus and nasal mucosal inflammation. In some embodiments, the composition may be used to address various damaging effects of sinus and nasal mucosal inflammation. The inflammation of the mucous membranes could be from food or environmental allergies, infection (viral, bacterial, fungal), laryngeal pharyngeal acid and/or bile reflux, chemical irritation (including side effects of nasally delivered medication), and climate change. The composition can help reduce the inflammation, whether it is acute or chronic.

In some embodiments, the composition may be used to soothe nasal/sinus pain or congestion by cleansing, moisturizing, and reducing inflammation in the nasal and sinus passages naturally and comfortably. When the composition is in the powder form, the powders are mixed with distilled water to form a nasal rinse solution. Rinsing the affected nasal passages using nasal rinse solution can also contribute to developing healthier and contaminate-free nasal tissue by improving mucociliary clearance and decreasing problematic inflammation, and providing a natural anti-microbial effect without the risk of antibiotic resistance. Using the nasal rinse solution can also contribute to improvements or reduction in the symptoms of chronic rhinosinusitis, allergic rhinitis, nasal dryness, diminished sense of smell, sinus headache, migraine headache, temporomandibular joint (TMJ) pain, and musculoskeletal pains.

In some embodiments, the composition may result in improvements or reduction in the symptoms of chromic rhinosinusitis, allergic rhinitis, nasal dryness, diminished sense of smell, sinus headache, migraine headache, TMJ pains, and/or musculoskeletal pain without the negative side effects. In some embodiments, the use of the composition may mitigate side effects of other medications.

One of the beneficial unexpected aspects included almost immediate (within 30-60 sec due to rapid transnasal mucosal delivery) results of improvement in stress and muscle tension. Also noted incidentally upon in-house testing was improvement in mood and cognition. The improvement in nasal inflammation and subsequent symptoms of nasal/sinus congestion and sinus pressure appeared to be comparable to traditional OTC intranasal steroid sprays, without the negative side effects of steroid absorption through the nasal mucous membrane. Range tested was 1 mg to 20 mg CBG per dose, and effects appeared to last longer with higher dosing, and cumulative benefits were noted. Anti-microbial benefits were also noted, with the added benefit of avoidance of antibiotic resistance.

### EXAMPLES

The following compositions were explored.

### Example 1

| | | Amount in 8 oz (29.57 mL) of |
|---|---|---|
| Compound | Amount (mg) | aqueous solution (mg/mL) |
| CBG | 1-20 | 0.033-0.676 |
| Polysorbate 80 | 20-80 | 0.238-0.676 |
| Xylitol | 1,000-4,000 | 33.818-135.272 |
| Sodium Chloride | 1,000-1,800 | 33.818-60.873 |
| Sodium Bicarbonate | 500 | 16.909 |
| Mullein Leaf Verbascum | 10-200 | 0.338-6.764 |

### Example 2

| | | Amount in 8 oz (29.57 mL) of |
|---|---|---|
| Compound | Amount (mg) | aqueous solution (mg/mL) |
| CBG | 1-20 | 0.033-0.676 |
| Polysorbate 80 | 20-80 | 0.238-0.676 |
| Xylitol | 1,000-4,000 | 33.818-135.272 |
| Sodium Chloride | 1,000-1,800 | 33.818-60.873 |
| Sodium Bicarbonate | 500 | 16.909 |
| Holy Basil Ocimum | 10-300 | 0.338-10.145 |

### Example 3

| | | Amount in 8 oz (29.57 mL) of |
|---|---|---|
| Compound | Amount (mg) | aqueous solution (mg/mL) |
| CBG | 1-20 | 0.033-0.676 |
| Polysorbate 80 | 20-80 | 0.238-0.676 |
| Xylitol | 1,000-4,000 | 33.818-135.272 |
| Sodium Chloride3 | 1,000-1,800 | 33.818-60.873 |
| Sodium Bicarbonate | 500 | 16.909 |
| Holy Basil Ocimum | 10-300 | 0.338-10.145 |
| Mullein leaf Verbascum | 10-200 | 0.338-6.764 |

### Example 4

| Compound | Amount (mg) | wt % |
|---|---|---|
| CBG | 5-15 | 0.10-0.30 |
| Polysorbate 80 | 40 | 0.790-0.791 |
| Xylitol | 3,000 | 59.23-59.35 |
| Sodium Chloride | 1,500 | 29.62-29.67 |
| Sodium Bicarbonate | 500 | 9.87-9.89 |
| Mullein Leaf Verbascum | 10 | 0.197-0.198 |

### Example 5

| Compound | Amount (mg) | wt % |
|---|---|---|
| CBG | 5-15 | 0.10-0.30 |
| Polysorbate 80 | 40 | 0.790-0.791 |
| Xylitol | 3,000 | 59.23-59.35 |
| Sodium Chloride | 1,500 | 29.62-29.67 |
| Sodium Bicarbonate | 500 | 9.87-9.89 |
| Holy Basil Ocimum | 10 | 0.197-0.198 |

What is claimed is:

1. A composition comprising cannabigerol, xylitol, and at least one of mullein leaf and holy basil, wherein the composition is for intranasal administration.

2. The composition of claim 1, wherein the cannabigerol is in an amount of about 1 mg to about 20 mg.

3. The composition of claim 1, wherein the composition further comprises a carrier.

4. The composition of claim 1, wherein the composition further comprises polysorbate 80.

5. The composition of claim 1, further comprising sodium chloride and/or sodium bicarbonate, and an emulsifier.

6. The composition of claim 1, wherein the composition further comprises an additional cannabinoid.

7. The composition of claim 1, wherein the composition further comprises cannabidiol.

8. The composition of claim 1, wherein the composition further comprises tetrahydrocannabinol.

9. The composition of claim 1, wherein the composition further comprises cannabichromene.

10. The composition of any one of claim 1, wherein the composition is in a powder form.

11. The composition of claim 10, wherein the composition is configured to form a nasal/sinus rinse.

12. The composition of any one of claim 1, wherein the composition is a nasal spray.

13. A method of alleviating symptoms of chronic rhinosinusitis comprising administering the composition of any one of claim 1 to a subject experiencing chronic rhinosinusitis symptoms.
